(19) United States
(12) Patent Application Publication
(10) Pub. No.: US 2020/0402665 A1
(43) Pub. Date: Dec. 24, 2020

(54) UNPLANNED READMISSION PREDICTION USING AN INTERACTIVE AUGMENTED INTELLIGENT (IAI) SYSTEM

(71) Applicant: GE Precision Healthcare LLC, Milwaukee, WI (US)

(72) Inventors: Min Zhang, San Ramon, CA (US); Gopal Avinash, San Ramon, CA (US); Yrjö Häme, Helsinki (FI); Ali Faisal, Helsinki (FI); Kevin Leung, San Ramon, CA (US); Jeff Hersh, Waukesha, WI (US)

(21) Appl. No.: 16/906,796

(22) Filed: Jun. 19, 2020

Related U.S. Application Data
(60) Provisional application No. 62/863,673, filed on Jun. 19, 2019.

(51) Int. Cl. G16H 50/20; G16H 10/60; G16H 50/30; G16H 40/20; G06Q 10/04; G16H 50/70; G16H 70/20; A61B 5/00; G06T 11/20

(52) U.S. Cl. CPC G16H 50/20; G16H 10/60; G16H 50/30; G16H 40/20; G06Q 10/04; G16H 50/70; G06T 11/206; A61B 5/7275; A61B 5/7264; A61B 5/746; A61B 5/7435; A61B 5/4842; G16H 70/20

(57) ABSTRACT

Techniques are described for predicting readmissions of patients to an inpatient healthcare facility. In an embodiment, a method comprises applying, by a system comprising a processor, applying, by a system operatively coupled to a processor, a readmission risk forecasting model to medical history data for a patient, wherein the readmission risk forecasting model comprises an attention-based graph neural network (A-GNN). The method further comprises, based on the applying, generating, by the system, a readmission risk score for the patient that reflects a probability of readmission of the patient following discharge from an inpatient healthcare facility. The method further comprises facilitating providing, by the system, the readmission risk score to at least one of the patient or a clinician involved in care of the patient.

### RELATED APPLICATION

This application claims priority to U. S. Provisional Application Ser. No. 62/863,67 filed Jun. 19, 2019 and titled “UNPLANNED ADMISSION PREDICTION USING AN INTERACTIVE AUGMENTED INTELLIGENT (IAI) SYSTEM,” the entirety of which application is incorporated herein by reference.

### TECHNICAL FIELD

This application generally relates to an interactive augmented intelligent (IAI) for predicting unplanned readmissions of patients to an inpatient healthcare facility.

### SUMMARY

The following presents a summary to provide a basic understanding of one or more embodiments of the invention. This summary is not intended to identify key or critical elements or to delineate any scope of the particular embodiments or any scope of the claims. Its sole purpose is to present concepts in a simplified form as a prelude to the more detailed description that is presented later. In one or more embodiments described herein, systems, computer-implemented methods, apparatus and/or computer program products are described that provide an interactive augmented intelligent (IAI) for predicting unplanned readmissions of patients to an inpatient healthcare facility.

According to an embodiment, a method can comprise applying, by a system operatively coupled to a processor, applying a risk model on application specific retrospective data from at least one source, wherein the risk model comprises an attention-based graph neural network (A-GNN). The method can further comprise, based on the applying, generating, by the system, an application specific risk score, and facilitating providing, by the system, the application specific risk score to one or more entities. In one or more embodiments, the risk model comprises a readmission risk forecasting model and the retrospective data comprises medical history data for a patient, the application specific risk score comprises a readmission risk score for the patient that reflects a probability of readmission of the patient following discharge from an inpatient healthcare facility, and the one or more entities comprise the patient or a clinician involved in the patients care.

In some embodiments, elements described in connection with the computer-implemented method scan be embodied in different forms such as a computer system, a computer program product, or another form.

### DESCRIPTION OF THE DRAWINGS

FIG. 1 presents a block diagram depicting the high-level architecture of an example system for reducing unplanned readmissions in accordance with one or more embodiments of the disclosed subject matter.

FIG. 2 presents example readmission risk forecasting model output data in accordance with one or more embodiments described herein.

FIG. 3 presents example similar case data that can be generated by the actional care plan module using historical care plan data in accordance with one or more embodiments described herein.

FIG. 4 presents a tale comparing different actionable care plans determined for patients with different characteristics and risks of unplanned readmission in accordance with one or more embodiments described herein.

FIG. 5 presents a high-level flow diagram of an example process for predicting and evaluating unplanned readmissions using an interactive augmented intelligent (IAI) system in accordance with one or more embodiments described herein.

FIG. 6 presents an illustration of an example outlier detection model in accordance with one or more embodiments described herein.

FIG. 7 presents an illustration of an example, the A-GNN based readmission risk forecasting model in accordance with one or more embodiments described herein.

FIG. 8 presents a block diagram of another example system for reducing unplanned readmissions in accordance with one or more embodiments of the disclosed subject matter.

FIG. 9 presents a high-level flow diagram of an example computer implemented method for reducing unplanned readmissions in accordance with one or more embodiments of the disclosed subject matter.

FIG. 10 presents a high-level flow diagram of another example computer implemented method for reducing unplanned readmissions in accordance with one or more embodiments of the disclosed subject matter.

FIG. 11 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding sections or in the Detailed Description section.

The disclosed subject matter provides systems, computer-implemented methods, apparatus and/or computer program products that facilitate predicting unplanned readmissions of patients to a healthcare system using an interactive augmented intelligent (IAI). In particular, the disclosed subject matter provides techniques to predict unplanned readmissions of patients to an inpatient healthcare facility (e.g., a hospital and skilled nursing facility (SNF), or the like) and adverse events following discharge from the inpatient healthcare facility (or another inpatient healthcare facility. In some implementations, the disclosed techniques are particularly directed to predicting unplanned readmissions within a defined timeframe of discharge (e.g., within 30 days of discharge).

In one or more embodiments, the disclose techniques leverage retrospective data of Medicare beneficiaries to predict unplanned admissions to hospitals or skilled nursing facilities (SNF). With these embodiments, the unplanned readmissions can be forecasted based on a data set comprising of Medicare administrative claims data, including Medicare Part A (hospital) and Medicare Part B (professional services). The disclosed technique can be used by a clinician for example when discharging the patient or interacting with a home-care patient. In this regard, the disclosed systems can facilitate the clinician in guiding high-risk patients to proper care and professional services in order to mitigate the risk of unplanned admissions to hospitals or SNFs. In addition, the system will recognize patients that fit a profile for recommending improved end-of-life care as an alternative to repeated, unnecessary and costly hospital visits.

In various embodiments, the system is directed to initially evaluating the most common causes of unplanned admissions. According to literature, the most common 30-day all-cause unplanned admissions for Medicare patients are: congestive heart failure, septicemia and pneumonia. These are also the most-costly conditions overall. The system aims to find the most common unplanned admission causes in the data and focus on such most common causes to prove the concept before expanding to a broader set of conditions. Additionally, this will provide a better understanding of the relative difficulty of prediction for different conditions and the corresponding feasible solutions, as not all causes of unplanned admission may be predictable based on historical data (e.g., admissions due to car accidents).

In various embodiments, the disclosed techniques employ one or more machine learning models/artificial intelligence models (e.g., deep learning models, neural network models, etc.) that employ a unique attention-based graph neural network (A-GNN), optimized to predict unplanned admissions. For each Medicare participant in the training data set, multiple samples are extracted along the timeline of the patient's Medicare program participation. For each training sample, one of the following three labels will be given: 1) positive, if an admission occurred within 30 days of prediction time (end-point of the sample) for the subset of conditions identified as most common; 2) neutral, if an unplanned admission occurred to a hospital or SNF within 30 days but not within the subset of conditions; and 3) negative, if no unplanned admission occurred. Only the cases predicted as positive will pass through the recommendation system for an action plan to reduce the unplanned admission risk.

Using the A-GNN model(s) the disclosed system can generate at least the following outputs: 1) the predicted unplanned admission risk score; 2) the patient characteristics including feature importance scores and their relationship, leading to the risk score as an explanation (useful primarily for high-risk patients); and 3) the recommendations for unplanned admission risk mitigations. These outputs will enable the clinician to effectively recognize the high-risk patients, understand the reasons for the high risk, and plan actions together with the patient to reduce the risk of unplanned admission, or to have a conversation on improved end-of-life care.

The disclosed techniques provide several innovative strategies and methodologies to bring the AI-derived predictions to front-line clinicians and patients to aid in providing appropriate clinical resources to model participants and to increase use of AI-enhanced data feedback for quality improvement activities among model participants. For example, the disclosed techniques provide innovations to explain AI at three levels: 1) at AI design level, 2) user interaction level, and 3) data feedback level.

Regarding the AI Design Level: 1), the disclosed systems provide a novel attention-based graph neural network (A-GNN) with feature importance score measurement. The A-GNN will provide the relationships and the importance score across features when making prediction. This will let the clinicians and patients know how the interactions across the features and how big an impact a given feature has on predicting the outcome. This will give the clinician the power of statistical analysis applied on the individual patient, to understand the factors that contribute to the patient's unplanned admission risk. 2) Based on the trained A-GNN, the disclosed systems will design an outlier detection model to alert the users when the prediction is not a confident one. This type of alert will ensure that predictions are more reliable and interpretable.

Regarding the User Interaction Level: 2), the disclosed systems can provide a web-based user interface to visualize the feature map graphs, the feature importance and the relationships of the features. The graph with features as nodes will enable users to explore the relationships across the features and their importance scores. The disclosed techniques further provide a recommendation system to present the similar cases in the historical database to support informed clinical decisions. In addition, the disclosed techniques provide a recommendation system to recommend actionable care plans to mitigate the risk of unplanned admissions. These recommendations may include a physical therapy, wound clinic, diabetes clinic, etc. Furthermore, the model facilitates providing a special case (end-of-life care plan). For example, in various embodiments, the model can identify patients with high unplanned admission risk, whose risk explanation profile matches with patients who have died within 6 months. For such patients, the clinician's recommendation list will include a discussion on improved end-of-life care as an alternative to standard hospital care. Studies have shown that most patients would prefer to die outside the hospital, but most nonetheless die in an intensive care environment. High-quality end-of-life care would have a positive impact in end-of-life quality and would save Medicare costly treatments so that resources can be used more effectively.

At the Data Feedback Level: 3), the disclosed systems can provide risk score prediction feedback and recommended actionable care plan feedback. In this regard, the discloses systems can incorporate a continuous learning framework to continuously/periodically retrain the model with new batches of the data added to the previous dataset with clinician's annotation/correction if the clinician chooses to do so. Predictions can be reviewed and confirmed by the clinician with reasoning score (importance score). If the clinician does not agree with the prediction and the reasoning, the case will send to the failure case database and reannotated for further model improvement. To enable a positive feedback loop in the model, the clinician can be asked to tell the system what action was recommended to each patient using a templatized format. Over time, these recommendations can be collected and used as additional training data for the recommendation component. The model will see which recommendations were in practice contributing to the improved outcomes. This will enable recommending the most effective care for each patient.

The disclosed system employs a novel AI network architecture comprising one or more attention based graph neural network (A-GNN) models to predict the risk of an unplanned admission given a patient's information from the available CMS data. GNNs are deep learning based methods that operate in the graph domain. The disclosed techniques provide a new GNN referred to herein as an “A-GNN,” which adds the attention module to the conventional GNN. Adding the attention module helps a conventional GNN add focus to the nodes/features in the graph, thereby producing the importance score of the features. More specifically, the A-GNN will assign a weighting to each parameter in the input feature space that will feed into the final outcome prediction. Features or subset of features will be rank-ordered based on the weighting in magnitude and leveraged for the prediction. In addition, the co-variance matrix of the graph will provide the feature relationship when making prediction. In this regard, in one or more embodiments, using an A-GNN, the system can classify patients as: 1) positive if an admission is predicted to occur within 30 days of prediction time (end-point of the sample) for the subset of conditions identified as most common; 2) neutral, if an unplanned admission occurred to a hospital or SNF within 30 days but not within the subset of conditions; 3) negative if no admission is predicted.

The disclosed system further employs an outlier detection model based on the trained A-GNN. In this regard, based on the trained A-GNN, an outlier detection model is developed and applied to determine (and alert clinicians) when the prediction is not a confident one. This type of alert will ensure that prediction results of the unplanned admission risk model are reliable and interpretable.

To further improve the model's interpretability, a similar historical case recommendation system model is also provided. The similar historical case recommendation system model can explore, extract and present similar cases and their outcomes in corresponding situations from a database to help clinicians determine the predictions when making the prediction of a new case.

The discloses techniques further provide an actionable care plan recommendation system. In this regard, after the risk score prediction, the proposed system and determine and recommend the related actionable care plan to the patient with a positive prediction. Then the clinician will determine the most appropriate action plan for the cases predicted as positive based on the actionable care plan recommendation system.

The disclosed techniques further employ continuous/periodical learning to optimize the various prediction models. For example, in various embodiments, the various AI algorithms described herein can be built with a closed-loop continuously learning enabled framework to continuously/periodically improve the performance of the models. In this regard, with respect to the risk score prediction model if the clinician does not agree with the prediction and the reasoning, the system can send the case to the failure case database, reannotated and leveraged for further model improvement. With respect to the actionable care plan recommendation model, the actionable care plan recommendations can be collected and used as additional training data for the recommendation component. The model will thus learn which recommendations were in practice contributing to the improved outcomes.

In various embodiments, the various features and functionalities of the disclosed systems can be built into a web-based application for ease of use and to provide immediate access and feedback for the clinicians.

Techniques are further provided to verify, validate, secure and control the proposed AI models. For example, in one or more embodiments, the data sets will be split into training, validation, data science test dataset and production test dataset to verify and validate the AI models. For the training and validation, a k-fold cross-validation approach will be used. Then the segregated test dataset that has never been used previously, will be evaluated to report the model performance. Finally, the reported performance of the model will be clinically validated using a production test dataset to re-check the generalizability of the model. All the trained models can further be encrypted and restful APIs will be built to access the models. All the development process will follow GEHC AI quality standard. Software V&V (verification and validation) process will be followed to check that our AI system meets specifications and that it fulfills its intended purpose. Unit tests, integration tests and component tests as well as the model consistency tests will be performed to ensure the quality control of the AI models.

The proposed model will work with clinicians and patients to explain AI-derived predictions in comprehensible and interpretable formats. Whenever an unplanned admission risk score is calculated, a profile with related features will be provided as explanation of the prediction. This profile will highlight the main characteristics in the patient's medical history that have contributed to the risk score.

1) Model are designed with Interpretability: In order to explain what the model has learned, the disclosed systems designed attention-based graph neural network (A-GNN) with feature importance score measurement. The A-GNN will provide the relationships and the importance score across features when making prediction. This will let the clinicians and patients know how the interactions across the features and how big an impact a given feature has on predicting the outcome.

2) Prediction results can be visualized interactively: In our system, the disclosed systems will build the web-based user interface to enable the interactions between users and the prediction results with contributing features and their scores. The graph with features as nodes will show in the web application enable user to explore the relations across the features and their importance score.

3) Recommendation system of similar cases helps clinicians make decision: When making the prediction of a new case, our AI system will explore and list the similar cases and their outcomes in corresponding situations from the database to help inform clinical decisions.

4) Recommendation system of actionable care plan mitigates the risk of unplanned admission: The proposed system will recommend the related actionable care plan to the patient with a positive prediction. Then the clinician will determine the most appropriate action plan for the cases predicted as positive based on the actionable care plan recommendation system.

5) Transparency—Model Design: the disclosed systems will design models with interpretability and explain ability. a) the disclosed systems will design A-GNN, which provides feature importance score and feature relationship. This will let the clinicians and patients know how the interactions across the features and how big an impact a given feature has on predicting the outcome. b) the disclosed systems will design an outlier detection model to alert the users when the prediction is not a confident one. This type of alert will ensure that predictions are more reliable and transparent.

6) Transparency—Result Visualization: In our system, the disclosed systems will build the web-based user interface to enable the interactions between users and the prediction results with contributing features and their scores. The graph with features as nodes will show in the web-application enable user to explore the relations across the features and their importance score.

4) Transparency—Similar Case Recommendation: the disclosed systems will design a recommendation system to present similar cases and their corresponding labeled ground truths to the clinicians. Such a system will help clinicians make decisions based on the historical data with enhanced confidence.

5) Transparency—Actionable Care Plan Recommendation: the disclosed systems will design a system to recommend related actionable care plans to patient cases with positive predictions. This system can help clinicians determine the most appropriate action plan.

The intended impact of our proposed solution falls into three key elements:

1) Designing and developing novel data-driven AI architectures to augment Human-AI collaborations and the explain ability of AI models.

2) Bringing the best industry/engineering development practices to support healthcare practices and deliveries including Medicare beneficiaries to reduce the risk of unplanned admission.

3) Incorporating clinician inputs in an active learning loop to continuously/periodically improve and expand the scope of original proposed solution, which may lead to new medical guidelines.

Additionally, the solution will have an immediate impact on empowering clinicians to recommend the proper care to their patients to reduce the risk of unplanned admission. Over time, the solution will outline the most effective guidance on patient activities. Such data-driven recommendations may lead to medical guidelines, as the model will follow the outcomes of giving the recommendations.

The nature of data-driven solution allows continuous learning with a growing, high quality, clinician curated dataset, and enables evolving and improving over time. Such an approach can help elevate the clinician to the next level with the provided explanations and recommendations.

The solution will manage potential adverse effects of automation and AI. First, the AI development process will follow industry best practices like Failure Mode and Effects Analysis (FMEA). Second, the potential adverse effects of the automation and AI will be managed with clinician's intervention. Although all the predictions and suggestion will be automatically populated by the AI, the predicted results will be reviewed and confirmed as a result of clinician's intervention. Third, the disclosed systems will build a corresponding outlier detection model to define the scope of the AI models. Any new input data that has different distribution/co-variate shift will be alerted to the user during the inferencing process indicating the results may not be as accurate as expected. Finally, a continuous learning framework with failure case database will be built to continuously/periodically improve the performance of the AI and to manage the potential adverse effects.

The disclosed solutions provide significantly improved techniques to predict and reduce unplanned hospital admissions and adverse events. Statistically significant relative difference in unplanned admission rates between patients that received the system's recommendation versus a control group that did not receive a recommendation. Cost savings resulting from decreased number of unplanned admissions. The 10 most common admission conditions resulted in over 9 billion dollars of cost for Medicare patients in 2011, and therefore even a small improvement of a few percent would save hundreds of millions of dollars. For monitoring technical performance: sensitivity, specificity, precision, positive and negative likelihood ratios of predicted risk scores. These scores are recorded during model development, and monitored throughout the operation of the model.

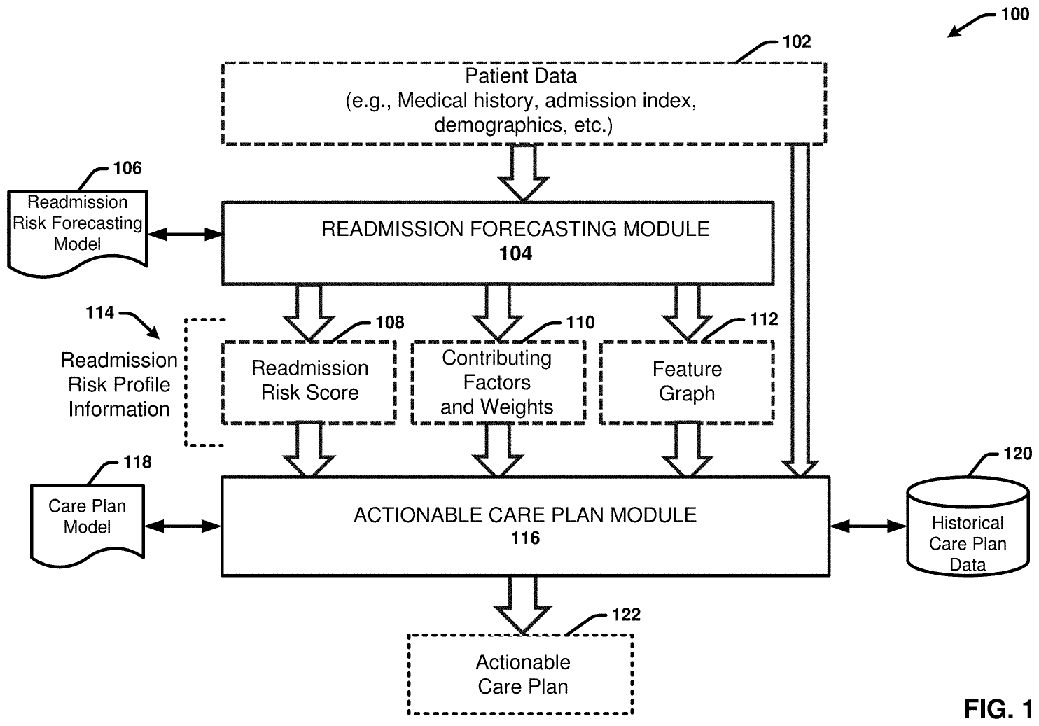

Turning now to the drawings, FIG. 1 presents a block diagram depicting the high-level architecture of an example system 100 for reducing unplanned readmissions in accordance with one or more embodiments of the disclosed subject matter. Elements described in connection with the disclosed system(s) and computer-implemented method(s) can be embodied in different forms such as a computer system, a computer program product, or another form.

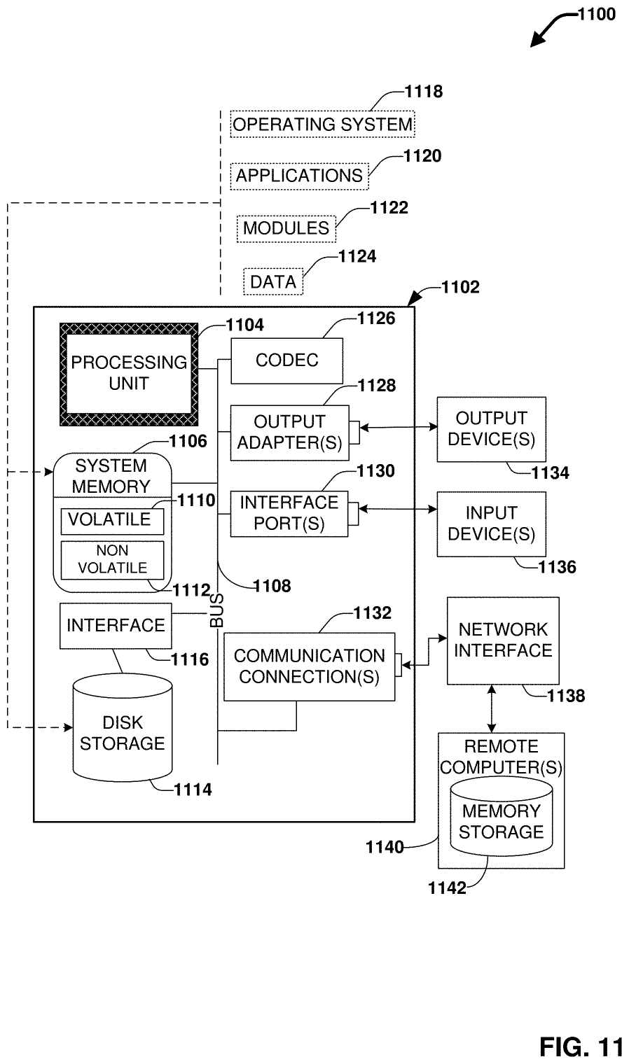

In this regard, one or more operations described with respect to systems and methods described herein can be performed by various types of computer systems comprising (or operatively coupled to) at least one process, and at least one memory, wherein the at least one memory stores executable instructions that, when executed by the processor, facilitate performance of described operations. For example, one or more of the operations described with reference to system 100 can be defined or otherwise embodied within one or more machine-executable components embodied within one or more machines (e.g., embodied in one or more computer readable storage mediums associated with one or more machines). Such components, when executed by the one or more machines (e.g., processors, computers, computing devices, virtual machines, etc.) can cause the one or more machines to perform the operations described. Examples of said processor and memory, as well as other suitable computer or computing-based elements, can be found with reference to FIG. 11 with respect to processing unit 1104 and system memory 1106, and can be used in connection with implementing one or more of the operations shown and described in connection with FIG. 1 or other figures disclosed herein.

System 100 can include a readmission risk forecasting module 104 and an actional care plan generation module 116. The readmission risk forecasting module 104 can facilitate forecasting readmission risk profile information for a patient that reflects a probability of readmission of the patient to an inpatient medical facility upon discharge using a readmission risk forecasting model 106. In particular, the readmission risk forecasting model 106 can comprise one or more machine learning models that have been trained to predict information regarding likelihood of readmission of a patient following discharge based on learned correlations in various factors associated with the patient's medical history, the patient's cause for admission (also referred to as the readmission index), demographic factors, and the like.

In various embodiments, the medical history data for a patient can include medical history information provided in by one or more electronic health record (EHR) databases and systems. For example, the patient medical history information can include internal medical history information for patients associated with a single healthcare organization, as well as medical history information aggregated for patients across various disparate healthcare organizations/vendors (e.g., internal and third-party organizations/vendors) and accessed via a healthcare information exchange system (HIE). Some clinical features included in the medical history data that can be used as input to the readmission risk forecasting model 106 can include but are not limited to: comorbidities, ongoing illnesses (including mental illnesses), past diagnoses, past hospital stays/admissions and associated information regarding past courses of care and length of stay (LOS), past intensive care unit (ICU) stays, past surgeries, regular and acute medications taken, and whether the patient has any implanted medical devices (IMDs) and if so, the type and location of the IMDs, exacerbation conditions associated to heart failure in last 6 months, historical total inpatient expenditure for the patient, historical total medical expenditures of the patient and the like.

The patient data 102 can also include information regarding their current admission from the time of admission to the time of discharge. In various embodiments, the current admission data can include initial admissions data, care progression data, and case worker data.

In this regard, the initial admissions can include known clinical information about a patient collected at or near the time of admission of the patient, including information regarding the context of admission (e.g., where, when, and why the patient was admitted), the state of the patient at or near the time of admission, and the initial clinical care ordered and/or provided to the patient at or near the time of admission. Some clinical features/factors included in the initial admissions data that can be used as input to the readmission risk forecasting model 106 can include but are not limited to: admission time, admission entry point (e.g., emergency room, elective, transfer, scheduled surgery, etc.), primary diagnosis/current condition, admission index, initial treatment provided (e.g., surgical procedures, diagnostic tests, medications administered, etc.), initial clinical orders, patient status and reported symptoms upon admission, initial care plan or care pathway prescribed, and the like. In some implementations, the initial admissions data can also include a measure of medical complexity, severity or risk determined for the patient which can be used as input to the respective care outcome forecasting models. The readmission risk forecasting module 104 can receive the initial admission data for a patient from various sources or systems. For example, the initial admissions data can be provided by and/or extracted from admission forms (e.g., filled out by the patient or another person accompanying the patient), from clinical data entry systems, from clinical notes (e.g., written, spoken and recorded, etc.), from electronic scheduling systems (e.g., providing information regarding scheduled procedures and clinical events), and the like.

The care progression data can include clinical information regarding a patient's status, location, and treatment over the course of the patient's stay. In this regard, the care progression data can provide a timeline of the patient's stay that tracks relevant information regarding the clinical treatment received and scheduled, the patient's status, and the patient's location (e.g., care unit) as a function of time. Some clinical features included in the care progression data that can be used as input to the readmission risk forecasting component 106 include but are not limited to: current diagnosis, current patient status, current patient location and duration at that location, medical treatment received (e.g., procedures performed, medications administered, etc.), clinicians involved in provision of the treatment (e.g., ordering physician, attending physician, nurses, etc.), laboratory tests conducted (e.g., including type, timing and results reported), imaging studies performed (e.g., including type, timing and results reported), other diagnostic tests performed, unit transfers, and occurrence of other defined medical events. In various embodiments, this care progression data can be reported and received in real-time over the course of the patient's stay. For example, this care progression data can be provided by and/or extracted from clinical data entry systems, from clinical notes (e.g., written, spoken and recorded, etc.), from electronic scheduling systems (e.g., providing information regarding scheduled procedures and clinical events), from clinical ordering systems, from medical imaging systems, from laboratory reporting systems, and the like.

The care progression data can also include tracked physiological parameters regarding a patient's physiological state (e.g., vital signs and other measurable physiological parameters) captured at one or more timepoints over the course of the patient's stay. In various embodiments, these physiological parameters can be received in real-time (or substantially real-time) from one or more medical monitoring devices, biofeedback devices and/or audio/visual monitoring devices.

The case worker data can include information provided by one or more case workers (or the like) that are involved with a patient's care. The case worker data can be received and extracted from notes, files, reports and the like provided by the case worker over the course of the patient's inpatient stay. For example, some patients, particularly complex needs patients, can be assigned a case worker to serve as a liaison for the patient and the different services they receive in and out of the hospital. Case workers can perform tasks including determining initial discharge plans and tracking and coordinating care activities, such as arranging dialysis for a patient post discharge. The case worker often works with the patient and the service provides in the community to coordinate and arrange these care activities. Case workers can also provide feedback regarding a patient's care needs, behaviors, capabilities (e.g., capabilities to care for oneself), and mental status. For example, a case worker can report information regarding specialty care requirements of a patient, such as whether a patient requires ventilator care, hemodialysis, chemotherapy, radiation therapy, wound vacuums, and/or has mental health care needs. In another example, a case worker can report information regarding a specialty diet of a patient, whether a patient requires medical shots to be administered, whether a patient has bandage change needs, and the like. Case workers further provide documentation regarding their involvement in the patient's care (e.g., as notes, activity logs, formalized reports, or the like). For example, case workers can provide documentation regarding their discharge plans, coordinated care activities, and observed patient care needs, behavior, capabilities, mental status, etc. In this regard, some clinical features included in the case worker data that can be extracted from the case worker documentation and used as input to the readmission risk forecasting model 106 can include but are not limited to: patient care needs, medical equipment needs, care activities scheduled, care activities performed, recommended discharge disposition, discharge activities scheduled, transportation arrangements, patient capabilities, patient mental status, patient behaviors, and the like.

In one or more exemplary embodiments, the readmission risk forecasting module 104 can group the patient data 102 for input to the readmission risk forecasting model 106 into the following three sets of risk factors: 1.) risk factors from patient data prior to first admission; 2.) risk factors related to past admissions; and 3.) risk factors related to the current admission. In some embodiments, this information can be exacted from various standard analytical files (SAF), insurance claims data files and the like associated with the patient in various electronic data sources.

The patient data 102 can also include various non-clinical patient factors the can influence or indicate a likelihood of readmission of the patient. In various embodiments, these non-clinical patient factors can include factors related to the patient's demographics, socioeconomics, personal patient support, and patient lifestyle.

In this regard, some example demographics factors that can be used as input to the readmission risk forecasting model 106 can include but are not limited to: patient age, gender, height, weight, body mass index (BMI), ethnicity/race, religion, language, marital status, nationality, birth location (e.g., country, state and/or city), and current residence location (e.g., country, state and/or city). Some example socioeconomic factors that can be used as input to the readmission risk forecasting model 106 can include but are not limited to: education level, occupation, income level per capita, median household income, debt, net worth, credit score, assets, home zip code, rural-urban community area (RUCA) code associated with the patient's current home location, criminal background (e.g., arrests, convictions, etc.), living family members (e.g., spouse, parents, grandparents, siblings, children, grandchildren), family member ethnicities, number of siblings, number of children, number of grandchildren, next of kin, emergency contact type, emergency contact person, and the like.

Information regarding personal patient support data can include information regarding who (if anyone besides that patient), will be responsible for caring for the patient from the point of discharge. This can include family, friends, case workers, or another individual (or group of individuals) that is hired or volunteered help. In some implementations, the personal patient support data can also include information regarding the patient's home environment or living arrangements (e.g., including type, structural features such as stairs/elevators, location, and other individuals that live there). The personal patient support data can also include factors regarding capabilities of the patient to care for oneself post-discharge. In this regard, some example, personal patient support factors that can be used as input to the readmission risk forecasting model 106 can include but are not limited to: whether the patient has anyone that will be responsible for the patient post-discharge, relationship of the person or persons responsible for the patient to the patient (e.g., friend, family member, type of family member), age of person or persons responsible for the patient, whether the patient has transportation, whether the patient lives alone, who lives with the patient (e.g., friends, family members, live in nurse, etc.), type of home environment (e.g., house, apartment), location of home environment, whether the home environment requires the patient to use stairs or an elevator, whether the patient is capable of performing daily life activities (e.g., feeding oneself, bathing oneself, clothing oneself, etc.), and mobility of the patient (e.g., ability to walk, requires a walker, requires crutches, requires a wheelchair, etc.).

Patient lifestyle data can include information regarding patient lifestyle activities and behaviors that can have an impact on the patient's medical condition or state during and after discharge. For example, some patient lifestyle factors that can be input to the readmission risk forecasting model 106 can include but are not limited to: frequency/amount of tobacco smoking, frequency/amount of alcohol use, frequency/amount and type of other recreational drug use, frequency/amount and type of exercise, recent foreign travel, and exposure to environmental pathogens through recreational activities or pets.

Below provides one example list of input data points that can be included in the patient data 102 and used as input to the readmission risk forecasting model.

Example Input Data Points:
Input risk factors from current admission:
   Demographics (age, sex, race etc.)
   procedure codes of procedures performed
   Date when the procedures are performed
   Care unit where patient is admitted to
   Location (home, hospice, nursing unit) where patient was previously discharged to (if applicable)
   current chief principal diagnosis and other diagnosis (e.g., as diagnosis codes)
   Diagnosis prior to admission (and information regarding whether the diagnosis is maintained)
   Cause of injury (e.g., using cause of injury code)
   Type and priority of admission with values (e.g., emergency, unknown, trauma etc.)
   For inpatient source for referral (e.g., from ER, from SNF, transfer from another hospital etc.)
   For outpatient reason of visit
   Claim type (institutional: inpatient, outpatient, SNF, Hospice, non-institutional: carrier)
   Amount paid by insurance entity;
   Payment amount due from beneficiary
   Total charges for all services in the institutional medical claim
   Type of service provided
   Facility that provided care, SNF, hospital etc.
   Medications provided
   Depending on type of service, a quantitative measure of service provided/performed
   Date of services performed
   Attending physician specialty
   Nature of billed service
   Medical equipment utilization data
   Type of tests performed (e.g., hematocrit or hemoglobin)
   Laboratory value for the most recent hematocrit or hemoglobin reading
Input risk factors from last admission:
   Patient status
   Type and priority of admission
   Source for referral
   Diagnosis
   Was each diagnosis present on admission
   Procedure codes
   Type of service provided
   Date when the procedures are performed
   Medical equipment utilization data

In this regard, at or near the time of discharge, the readmission risk forecasting module 104 can receive patient data 102 comprising at least some of the various factors and data points described above input to the readmission forecasting model 106. The readmission risk forecasting module 104 can further apply the readmission risk forecasting model 106 to the input data to generate readmission profile information 114 for the patient.

It should be appreciated that historical patient data for past patients corresponding to the above described patient data 102 can be used to train and develop the readmission risk forecasting model 106. However, in addition to the above described patient data 102, the training data used to train the readmission risk forecasting model 106 can also include post-discharge information tracked for the patients following discharge, including information regarding if they were readmitted and if so, when, where and reason for readmission. The post-discharge information can also include information regarding the health of the respective patient post discharge, including information regarding positive and negative outcomes, adverse reactions and the like. The post-discharge information can also track end-of-life, including timing of death of post discharge patients, cause of death, location of death, and the like. In some embodiments, this post-discharge information can be included in the historical care plan data 120.

In one or more embodiments, the readmission risk forecasting model 106 can be or comprise a unique attention-based graph neural network (A-GNN). GNNs are deep learning-based methods that operate in the graph domain. The disclosed A-GNN adds an attention module to the conventional GNN. The attention module provides for added focus on the nodes/features in the graph and produces importance score of the graph features. More specifically, the disclosed A-GNN assigns a weighting to each parameter in the input feature space that will feed into the final outcome prediction. As applied to the readmission risk forecasting problem, the A-GNN can assign weights (also referred to as importance scores) to various input factors/features extracted from the patient data that represents their degree of contribution to the patient's probability of readmission. The A-GNN can further rank and order factors/features or subset of features on the weighting in magnitude and employ the weighting scheme for predicting likelihood of readmission within a defined timeframe (e.g., 30 days). In addition, the A-GNN model can generate a co-variance matrix of the graph that identifies relationships between two or more different factors/features with respect to how they contribute to the patient's probability of readmission.

In the embodiment shown, the readmission risk profile information 114 that can be output/generated by the readmission risk forecasting model 106 can include a readmission risk score 106, one or more contributing factors and weights 110, and a feature graph 112. The readmission risk score can comprise a value that reflects an expected probability of readmission of the patient determined using the readmission risk forecasting model 106. The scoring method/scale employed for the readmission risk score 108 can vary. In some embodiments, the readmission risk score can comprise a percentage score (e.g., from 0-100%), wherein the higher the score, the higher the probability of readmission.

In another example embodiment, the readmission risk score 108 can employ a binary scoring valuation, wherein a patient is classified as either likely to be readmitted or not likely to readmitted. Additionally, or alternatively, the readmission risk score 108 can classify patients as being unlikely to be readmitted, being neutral, or being likely to be readmitted. For example, in one embodiment, the training data set for training the readmission risk forecasting model can include pre and post discharge information for various patients with different clinical case factors, patient demographics, lifestyle factors and the like. The training data for each patient can be extracted along the timeline of the patient's medical history prior to discharge and up to a defined time point following discharge (e.g., 6 months, 1 year, 2 years, 5 years, until end-of-life, etc.). In one implementation, for each training sample, one of the following three labels will be given: 1) positive, if a readmission occurred within 30 days of prediction time (end-point of the sample) for the subset of conditions identified as most common; 2) neutral, if an unplanned admission occurred to a hospital or SNF within 30 days but not within the subset of conditions; and 3) negative, if no unplanned admission occurred. In some implementations as described in greater detail below, only the cases predicted as positive can be passed through the actionable care plan module for an action plan to reduce the unplanned readmission risk.

The contributing factors and weights 110 can identifying the relevant contributing factors (and/or factor values) that impact the patient's readmission risk score (e.g., used to calculate the patient's readmission risk score) determined by the readmission risk forecasting model 106. The feature graph 112 can comprise an A-GNN generated graph comprising nodes corresponding to the respective contributing factors with connections (e.g., lines/edges) between the nodes (or subsets of the nodes) that represent the relationships between the factors. In particular, as discussed above, the A-GNN architecture of the readmission risk forecasting model 106 can identify various factors included in the patient's data that are relevant to predicting the patient's probability of readmission. The A-GNN model can further determine importance scores for the identified factors that reflect their relative importance to the patient's probability of readmission. The A-GNN can further rank and order these factors with their weights associated therewith and output this information as contributing factors and weights 110. In this regard, by using an A-GNN architecture, the readmission risk forecasting model 106 can provide the relationships and the importance score across features when making readmission risk predictions. This will let the clinicians and patients know how the interactions across the features and how big an impact a given feature has on predicting the outcome. This will give the clinician the power of statistical analysis applied on the individual patient, to understand the factors that contribute to the patient's unplanned admission risk.

As noted above, in some embodiments, the patient data 102 can include information regarding the primary reason (or reasons) or cause of admission of the patient to the inpatient facility from which the patient is being discharged. This reason or cause for admission is generally referred to as the admission index. In various embodiments, the contributing factors and weights 110 data can specifically identify or call out the index of admission for the patient as this factor has been generally found to have strong correlation to the patient's readmission probability. However, it should be appreciated that a variety of factors included in the patient data 102 can impact a patient's readmission risk profile.

In addition, in some embodiments, the contributing factors and weights output can identify and highlight a predicted cause of readmission in implementations in which the readmission risk score 108 indicates that patient is likely to be readmitted (e.g., based on the score being above a threshold or otherwise satisfying a readmission criterion). With these embodiments, the readmission risk forecasting model 106 can further be configured to predict a cause for readmission. In some implementations, the readmission forecasting module 104 can be configured to determine the predicted cause based on a determination that the risk score is classified as high (relative to a set threshold). The readmission forecasting module 104 can determine the risk factors and/or predicted cause from the patient's data as explanations to the high risk score. For example, adverse events are one of the main contributing factors. The predicted causes for unplanned admission can be focused on the most common conditions as found in the data. Amongst others, the disclosed systems expect these to include the following three causes, identified as the most common and also most costly causes of readmission for elder patients: congestive heart failure (CHF), septicemia, and pneumonia.

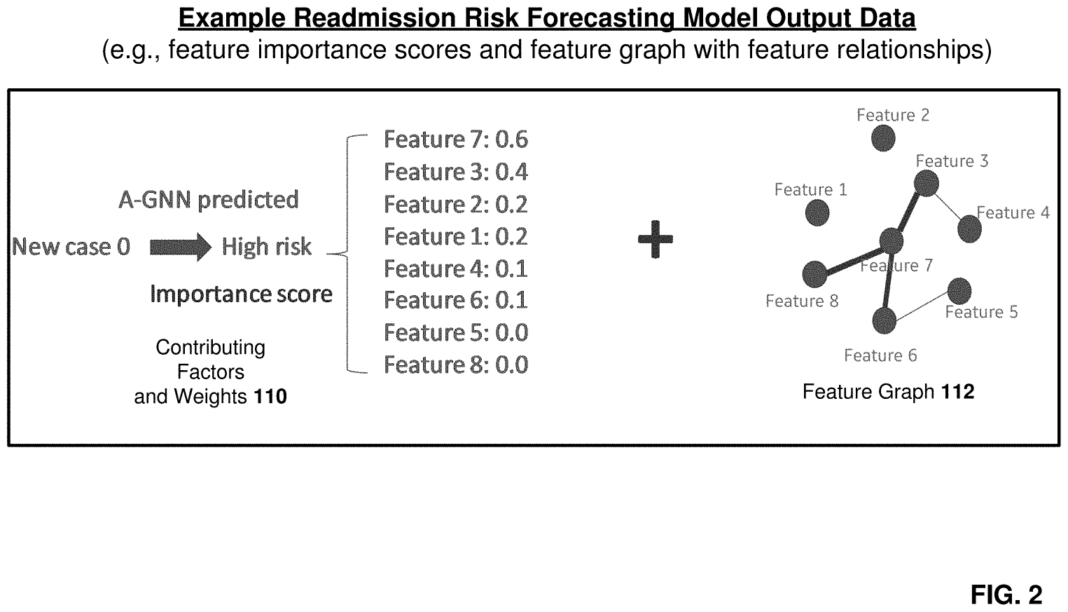

FIG. 2 presents example readmission risk forecasting model output data in accordance with one or more embodiments described herein. In the embodiment shown, the output data for a new patient case identified in FIG. 2 as “new case 0” includes a risk classification of high risk. In accordance with this example, the risk classification corresponds to the readmission risk score 108. The output data further include information regarding identified contributing features and their determined importance scores (e.g., which corresponds to the contributing factors and weights 108). The contributing features are respectively identified as features 1-8. The features are further order from top to bottom according to their important scores, highest to lowest. The output data further includes a feature graph 110 generated by the A-GNN architecture of the readmission risk forecasting model. As shown in FIG. 2, the feature graph 110 provides mappings between features that have relationships with other features. Those nodes/features without connections (e.g., feature 1 and feature 2) are standalone features. The nodes with connections to more than one other node are further indicated with darker (thicker) connections lines. At high level, one can look at feature graph 110 and see that feature 7 has the most connections and thus would be assumed to play a strong role in the case being classified as high risk. The is the case, as reflected by feature 7 having the highest importance score.

With reference again to FIG. 1 in various embodiments, the actionable care plan generation module 116 can determine and recommend an actionable care plan 122 for reducing the probability of readmission (or otherwise mitigating readmission) based on a determination that the readmission risk score 108 reflects a high probability of readmission (e.g., based on the score being above a threshold score, based on the score being a classification as positive, or the like). With these embodiments, the actionable care plan module 116 can determine the actionable care plan based on the readmission profile information 114, the patient data 102 and historical care plan data 120. In some implementations, the actionable care plan can further use one or more machine learning models to determine the actionable care plan 122 based the patient data 102, the readmission risk score 108, the contributing factors and weights 110 (e.g., including the predicted cause for readmission), and the historical care plan data 120. In the embodiment shown, these one or more machine learning models are represented by care plan model 118.

In this regard, the historical care plan data 120 can comprise historical action plan data identifying action plans that resulted in positive outcomes that were performed for other patients. For example, the historical care plan data can identify various defined clinical actions and/or courses of care (e.g., physical therapy, wound clinic, diabetes clinic, etc.) that were performed for discharged patients in the past with different clinical cases and characteristics that resulted in the patients not being readmitted or reducing an amount of time until the patients were readmitted. The historical care plan data 120 can also include patient data for the other patients comprising same or similar information as the patient data 102 described above, as well as information regarding reason for discharge, status at the time of discharge, and the like. In some implementations, the historical care plan data 120 can also include readmission risk profile information determined for the patients. For example, in addition to the action plans performed for the respective historical patients that resulted in preventing or mitigating their readmission, the historical care plan data 120 can also identify their predicted readmission risk score, contributing factors and weights, and predicted cause of readmission.

In this regard, in some embodiments, based on a determination that a patient's readmission risk is high (e.g., based on the patient's readmission risk score 108), the actionable care plan module 116 can access the historical care plan data 120 to identify historical cases for other patients that are similar to the patient's case. For example, the actionable care plan module 116 can identify cases that are similar to the patient's case based on those patients having readmission risk profiles with a defined degree of similarity to the patient's readmission risk profile (e.g., similar readmission sores, similar contributing factors and/or weights, similar predicted causes for readmission, etc.). Additionally, or alternatively, the actionable care plan module 116 can identify cases that are similar to the patient based on those patients having similar patient data (e.g., similar medical history data, similar admission index data, similar demographic data, etc.).

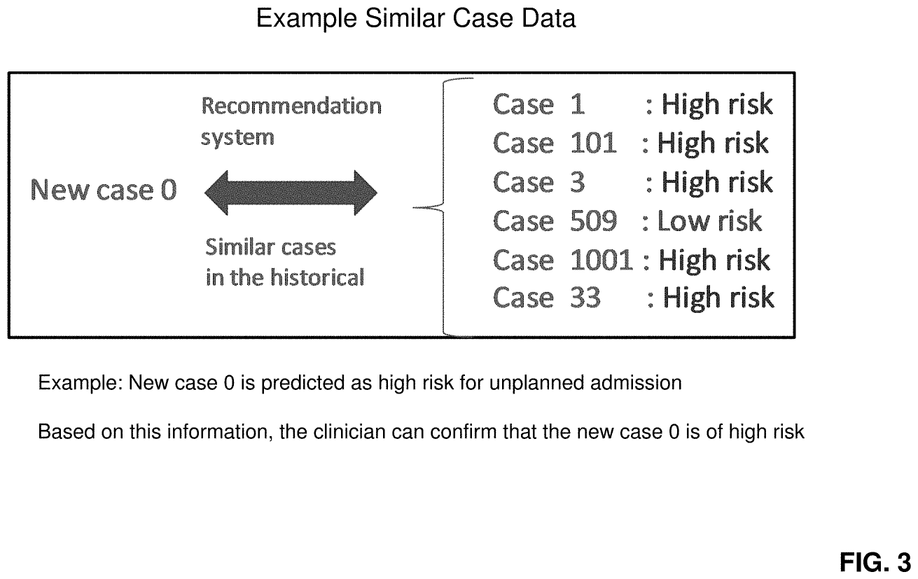

FIG. 3 presents example similar case data that can be generated by the actional care plan module 116 using historical care plan data 120 in accordance with one or more embodiments described herein. In the example shown in FIG. 3, the new patient case being evaluated is again referred to as “new case 0”. Continuing with the example output data shown in FIG. 2, the actional care plan module 116 can identify similar cases to “new case 0” in the historical care plan data 120 using defined similarity criteria with respect to the similarities between their patient data (e.g., medical history, admission index, demographics, etc.) and/or the readmission profile information (e.g., contributing factors and weights 110, predicted cause of readmission, etc.). Each of the similar cases can also include their determined readmission risk score, which in this example is a binary classification as either being high risk or low risk. In this example, six similar cases were identified, and 5 out of the 6 case were classified as being high risk, thus confirming that “new case 0” is likely high risk as well.

With reference again to FIG. 1, In some embodiments, the actionable care plan module 116 can further evaluate the similar actionable care plans performed for the similar cases to determine the most appropriate actionable care plan for the current patient. Additionally, or alternatively, the analysis can be performed or facilitated using a care plan model 118. In this regard, the care plan model 118 can be or comprise one or more machine learning models trained on the historical care plan data 120 and configured to output a recommended actionable care plan 122 based on an input data set comprising the readmission risk profile information 114 and/or one or more defined factors included in the patient data 102.

In various embodiments, the outputs of the readmission forecasting module 104 and/or the actionable care plan module 116 can be provided to one or more end users (e.g., the patient, a clinician, a system administrator, etc.), via a suitable user interface or graphical user interface (GUI). As used by clinicians, these outputs will enable the clinicians to effectively recognize the high-risk patients, understand the reasons for the high risk, and plan actions together with the patient to reduce the risk of unplanned admission, or to have a conversation on improved end-of-life care.

In some embodiments, these outputs can be rendered and presented to an end-user via a web-based user interface. For example, in one or more embodiments, the interactive GUI can generate and present an interactive version of the feature graph 112 with nodes of the graph corresponding to the contributing factors and connections between the nodes indicating the relationships between the features. The interactive version of the feature graph can allow users to select respective nodes and connections to view and explore the relationships across the features and their importance scores.

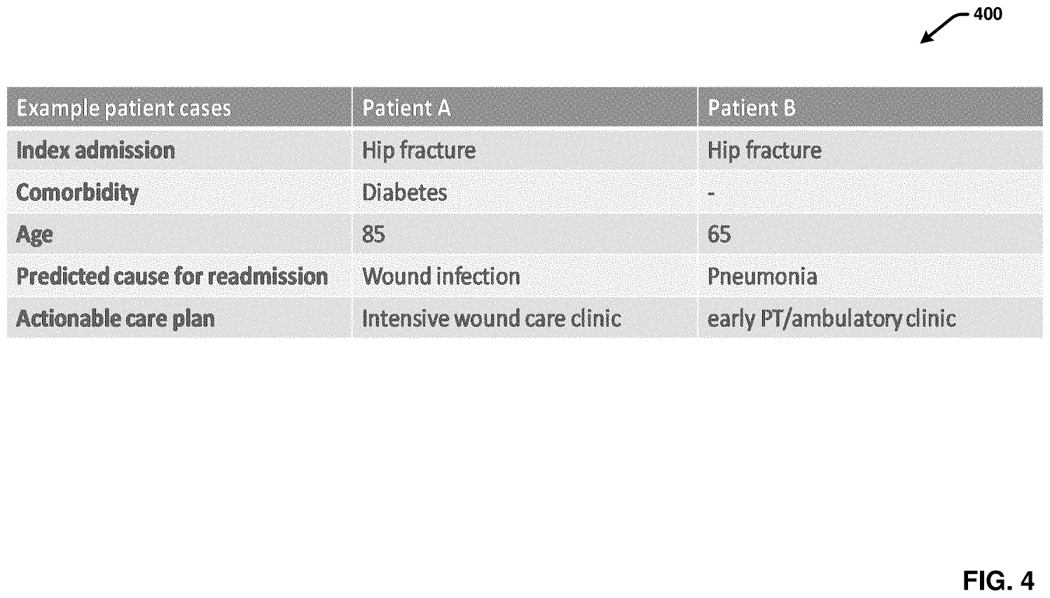

FIG. 4 presents a tale 400 comparing different actionable care plans determined for patients with different characteristics and risks of unplanned readmission in accordance with one or more embodiments described herein. In particular, table 400 compares two example patient cases with same index admissions (hip fractures) and different readmission profiles as determined based on their individual patient data. As shown in table 400, patient A has a comorbidity of diabetes and is 85 years old, while patient B has no comorbidly and is only 65 years old. Based on these example variances between the patients, the predicted causes of readmission for patient A and patient B are different (e.g., wound infection and pneumonia, respectively). Accordingly, the actionable care plans for the respective patients are also different.

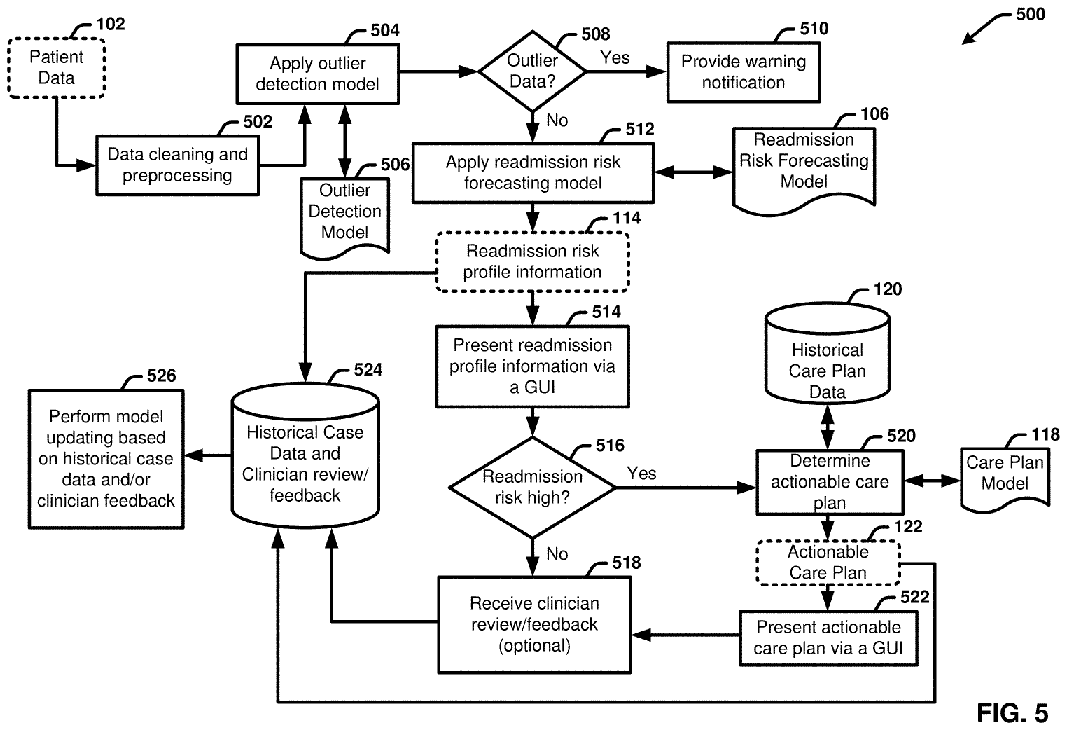

FIG. 5 presents a high-level flow diagram of an example process 500 for predicting and evaluating unplanned readmissions using an interactive augmented intelligent (IAI) system in accordance with one or more embodiments described herein. In various implementations, process 500 can be performed by system 100 (and additional systems described herein).

In accordance with process 500, at 502 the system can receive patient data 102 for a patient (e.g., at or near the time of discharge) and initially perform data cleaning and data pre-processing. For example, the data cleaning and pre-processing can involve identifying and extracting the relevant features/factors (and values) included in the patient data that can be used as input to the readmission risk forecasting model 106 (and optionally the care plan model 118). In this regard, the data cleaning and preprocessing at 502 builds an indexed list of features and feature vales.

In the embodiment shown, at 504, before the patient data is input to the readmission risk forecasting model 106, the input list of features can be processed using an data outlier detection model 506 to determine whether the patient data is within the scope of the readmission risk forecasting model's inferencing capability. In this regard, the outlier detection model 506 can be used to determine whether the received patient data is within a scope of training data used to train the readmission risk forecasting model 106 to ensure that there is no co-variate shift of the new data comparing to the training dataset (e.g., to determine whether the input data is within the scope of the training data used to develop the unplanned admission risk model). In various embodiments, the outlier detection model 506 can also be or include an A-GNN. If at 508, the patient data is detected as an outlier (e.g., outside the scope of the), then at 510, the system can generate notification indicating that the level of confidence in the accuracy of the predictions generated by the readmission risk forecasting model 106 on the patient data 102 is low.

At 506, if the patient data is not detected as an outlier (and/or the clinician would still like to see the results of the readmission risk forecasting model 106 despite the low confidence notification), then at 512, the system can apply the readmission risk forecasting model 106 to the patient data to generate the readmission risk profile information 114 (e.g., a predicted readmission risk score 108 representative of a predicted risk (probability) of unplanned readmission, as well as information identifying the relative importance/contribution of contributing features to the predicted readmission score 108, and information describing or indicating the relative relationships between the contributing features, such as a feature graph 112. In various embodiments, at 514, the readmission risk profile information 114 can be presented to one or more entities (e.g., clinicians) through an interactive user interface (UI). The readmission risk profile 114 (along with the corresponding patient data 102) can also be stored in a database for use future model updating and optimization. In the embodiment shown, this database 524 can comprise historical case data and clinician review/feedback data.

In some embodiments, at 516, the system can further determine if the readmission risk is high based on the readmission risk score 108 included in the readmission risk profile information 114. Based on a determination that the readmission risk is not high, the case data (e.g., the patient data and/or the readmission risk profile information 114 can be added to database 524. At 518 the clinician can also (optionally) review and annotate the readmission risk profile information 114, providing feedback regarding the accuracy of the readmission risk profile information 114.

If however at 516, the system determines that the readmission risk is high, then at 520, the system can determine an actionable care plan 122 for preventing or minimizing the occurrence/risks of the unplanned readmission using the historical care plan data 120 and/or a care plan model 118. For example, based on the readmission risk profile information 114 and the patient data 102, the system can search the historical care plan data 120 for historical patient cases (provided in an accessible database) that are similar to the patient's case. In one embodiment in which the readmission risk profile information comprises a feature graph and the historical care plan cases also comprise feature graphs, the system can find similar cases based on the distances between two feature graphs. In various embodiments, the system (e.g., the actionable care plan generation module 116) can determine the actionable care plan using example care plans determined for same or similar cases (e.g., based on the patient, the predicted cause of readmission, the contributing factors, and the like) stored in the actionable care plan database to reduce the risks of unplanned admission. At 522, the system can present the actionable care plan 122 via a GUI which can be access and reviewed by a clinician (or another suitable entity) at 518. In some implementations, the identified similar cases can also be presented to the clinician in the interactive UI to help the clinician make a final decision regarding whether the clinician agrees or disagrees with the output results of the unplanned admission risk model.

In this regard, at 518 the clinician can evaluate and annotated the model predications and actionable care plan and provide feedback regarding the accuracy of the models output. In some implementations, if the clinician confirms the model's prediction is correct and the case is predicted NOT as positive, the system will make the case as correct prediction without additional recommended actionable plan as with routine care recommendations. If, however at 518, the clinician confirms the model's prediction is incorrect, the system can annotate those inaccurate as stored in database 524. At 526, the system can periodically retrain the readmission risk forecasting model 106, the outlier detection model 506 and/or the care plan model 118 based on the data collected in database 524 and the collected clinician feedback/annotations.

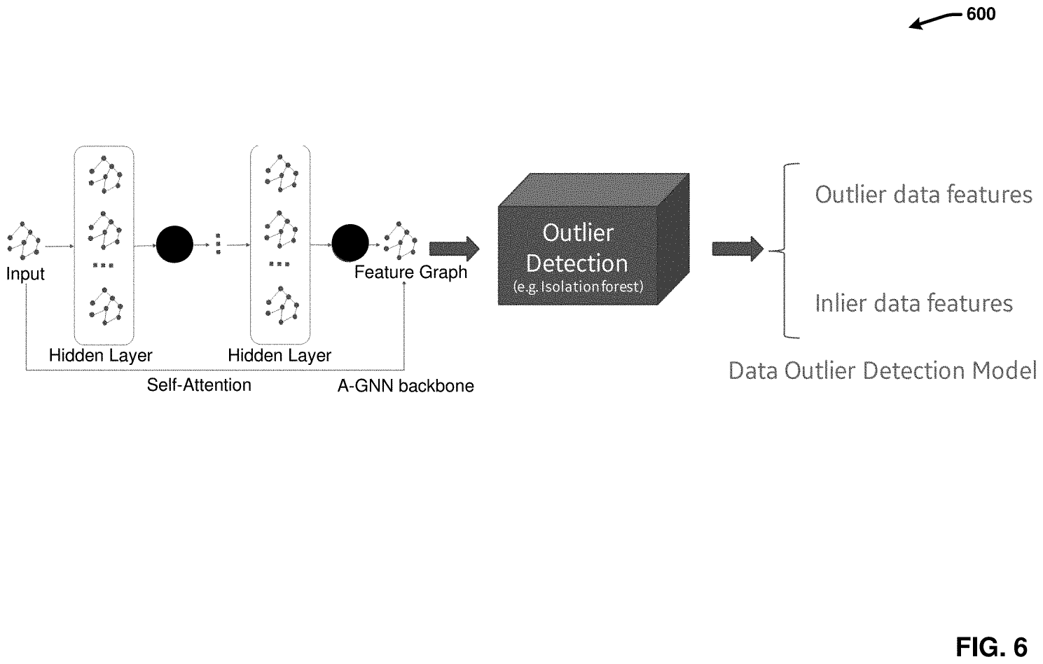

FIG. 6 presents an illustration of an example outlier detection model 600 (e.g., outlier detection model 506) in accordance with one or more embodiments described herein. In the embodiment shown, the outlier detection model 600 can comprise an A-GNN backbone combined with an outlier detector (e.g., an isolation forest detector or the like). In accordance with the embodiment shown, training data can be fed into a trained A-GNN model to extract the features graphs for every instance. Based on the extracted features, the system can build an outlier detection model to detect whether an instance of a new input data set (new patient data) is an outlier or inlier. If it is an outlier, this provides an indication that the system has a low degree of confidence that the unplanned admission risk model will generate an accurate prediction.

In one or more embodiments, training for the outlier detection model 400 can proceed as follows:

1. After A-GNN trained, all the training dataset will pass through to the A-GNN model to extract the feature graphs for training dataset.

2. Based on the feature graph information from training dataset, an unsupervised outlier detection model (isolation forest model) will be trained at different threshold values (0.1-0.4).

3. The test dataset will pass through the trained A-GNN model to extract feature graphs for test dataset and to evaluate the outlier detection model.

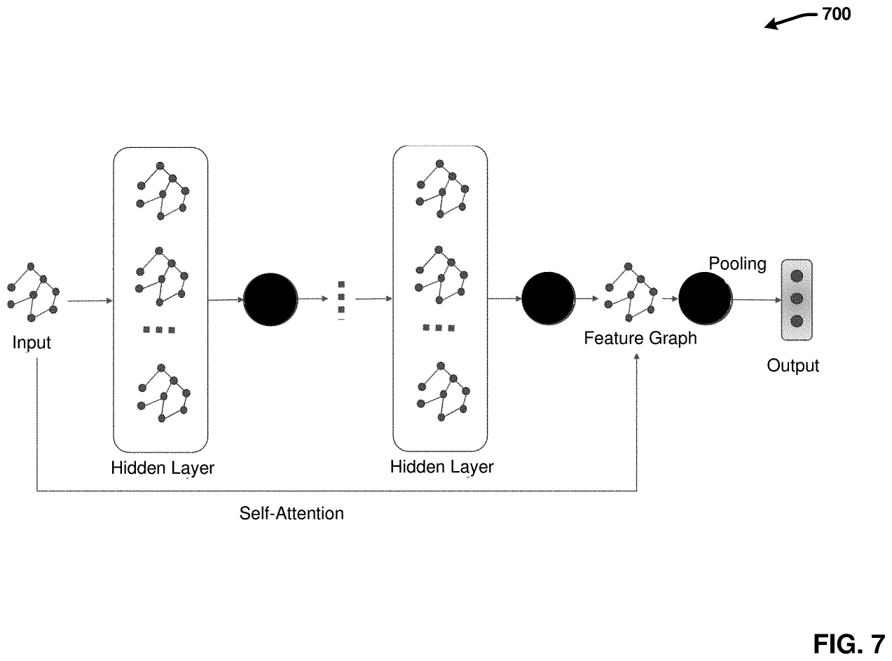

FIG. 7 presents an illustration of an example, the A-GNN based readmission risk forecasting model 700 (e.g., readmission risk forecasting model 106) in accordance with one or more embodiments described herein.

In one or more embodiments, the A-GNN based readmission risk forecasting model 700 takes the concatenation of graph input of feature matrix and feature adjacent matrix to predict unplanned admission risk score (high, neutral, low). Each node represents a feature from patient claim data. The network will automatically learn a feature graph indicating which features contribute most to the output prediction with attention module. The feature matrix of feature graph indicating the relationship when making the prediction.

In one or more embodiments, training for the readmission risk forecasting model 700 can proceed as follows:

1. The patient's claim data will be cleaned, processed (removing/filling the missing data, removing outliers/extreme data, standardization etc.) to generate the datasets used for training, validation, data science testing and production testing. The production testing dataset will be fully segregated from the other datasets.

2. Datasets for training, validation will be re-organized into k-fold cross validation datasets. On-the-fly data augmentation will be implemented to train the A-GNN network. Sensitivity, specificity, precision and AUC of the ROC will be calculated to evaluate the performance of the A-GNN. The best A-GNN model will be selected based on the validation dataset.

3. The model will be tested using the Data science test dataset to report the final performance and the segregated production test dataset will be evaluated once to finally validate the reported performance.

With reference again to FIG. 1, in various embodiments, at care plan model 118 can also employ an A-GNN based model to facilitate determining the similar cases. In this regard, the care plan model 118 training and development can proceed as follows:

1. After A-GNN trained, all the training dataset will pass through to the A-GNN model to extract the feature graphs for training dataset.

2. All the feature graphs will be stored in the case study databased with ground truth.

3. The test dataset will pass through the trained A-GNN model to extract the feature graphs for test dataset and several graph distance metrics will be used to calculate the pairwise distances for each test example to all training examples, and best distance metrics will be selected as the distance measurement for the recommendation system.

The various models discussed herein (e.g., the outlier detection model 506, the readmission risk forecasting model 106 and the care plan model 118) can be periodically retrained using a closed loop retraining process. In this regard, the system can periodically retrain the models discussed herein based on the new data and the feedback collected and annotated, with more weights added to the cases in the failure case database (oversampling the failure cases).

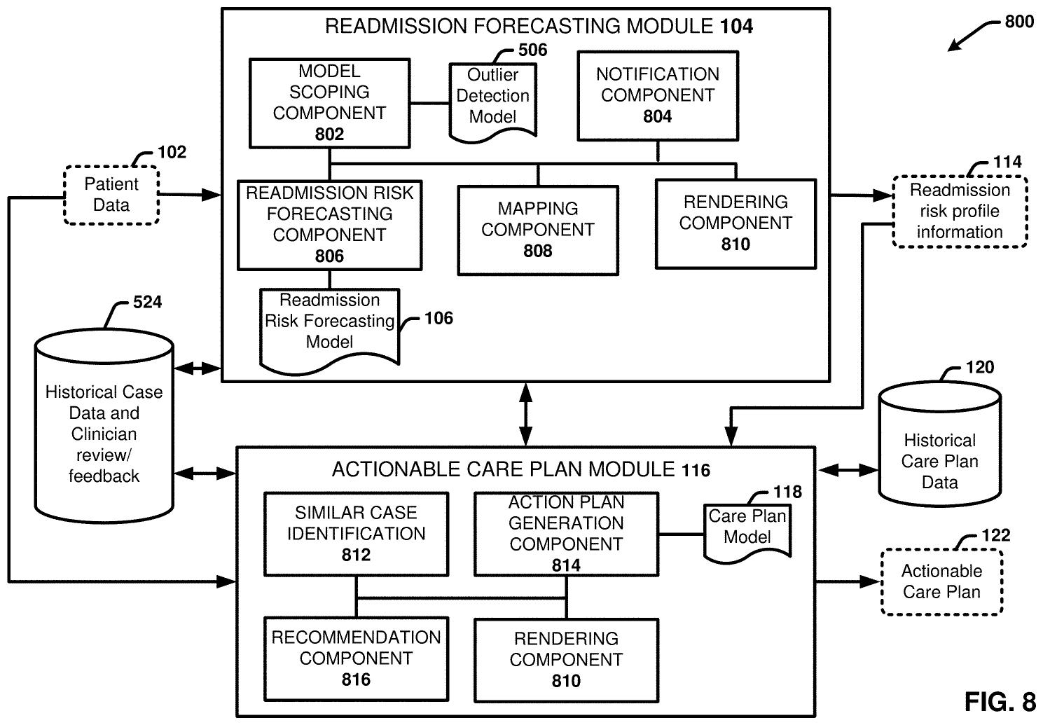

FIG. 8 presents a block diagram of another example system 800 for reducing unplanned readmissions in accordance with one or more embodiments of the disclosed subject matter. Repetitive description of like elements employed in respective embodiments is omitted for sake of brevity.

System 800 includes same or similar components introduced with reference to FIGS. 1 and 5, with some additional computer-executable components that can perform various specific operations described with reference to FIGS. 1 and 5 and process 500.

With reference to FIG. 8 in view of FIGS. 1 and 2, system 800 can include a readmission risk forecasting component 806 that can apply the risk forecasting model 106 to medical history data for a patient (e.g., included in patient data 102) and generate readmission risk profile information 114 for the patient. System 800 further includes a rendering component 810 that facilitates providing the readmission risk score to at least one of the patient or a clinician involved in care of the patient (e.g., at a device via a GUI).

System 800 further includes a mapping component that can generate an interactive feature graph (e.g., feature graph 112) comprising nodes respectively corresponding to the factors and connections between the nodes representing the relationships, and wherein the rendering component further facilitates rendering the interactive feature in a network accessible graphical user interface. System 800 can also include a model scoping component 802 that can apply the outlier detection model 506 to the patient data 102 to determine whether the patient data 102 is within a scope of training data used to train the readmission risk forecasting model, and a notification component 804 that can generate the warning notification based on a determination that the medical history data is outside the scope of the training data.

System 800 can also include a recommendation component that 816 that can recommend the actionable care plan 122 for reducing the probability of readmission based on a determination that the readmission risk score reflects a high probability of readmission. System 800 can also include a similar case identification component 812 that identifies, in one or more databases (e.g., historical care plan data 120 database), historical action plan data identifying action plans that resulted in positive outcomes that were performed for other patients having readmission risk profiles with a defined degree of similarity to the readmission risk profile for the patient. System 800 can also include an action plan generation component 814 that determines the actionable care plan 122 based on the historical action plan data 120, and in some implementations, using the care plan model 118. In some embodiments, the similar case identification component 812 further identifies the historical action plan data based on the other patients having similar medical health histories to the patient. In the embodiment shown, the actionable care plan module 116 can also comprise the rendering component 810 to facilitate rendering the actional care plan 122 to one or more entities via a suitable GUI.

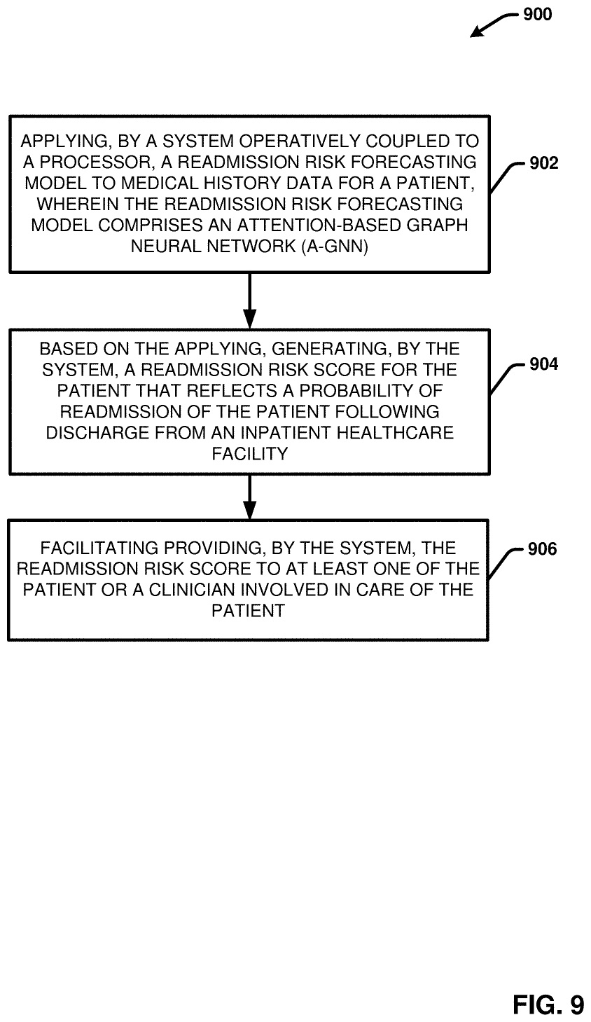

FIG. 9 presents a high-level flow diagram of an example computer implemented method 900 for reducing unplanned readmissions in accordance with one or more embodiments of the disclosed subject matter. Repetitive description of like elements employed in respective embodiments is omitted for sake of brevity.

At 902, a system operatively coupled to a processor (e.g., system 100, system 800, or the like), can apply (e.g., using readmission risk forecasting component 806) a readmission risk forecasting model (e.g., readmission risk forecasting model 106) to medical history data for a patient (e.g., patient data 102), wherein the readmission risk forecasting model comprises an A-GNN. At 904, based on the applying, the system can generate (e.g., (e.g., by the readmission risk forecasting component 806) a readmission risk score (e.g., readmission risk score 108) for the patient that reflects a probability of readmission of the patient following discharge from an inpatient healthcare facility. At 906, the system can facilitate providing (e.g., via the rendering component 810) by the system, the readmission risk score (e.g., as included in the readmission profile information 114) to at least one of the patient or a clinician involved in care of the patient.

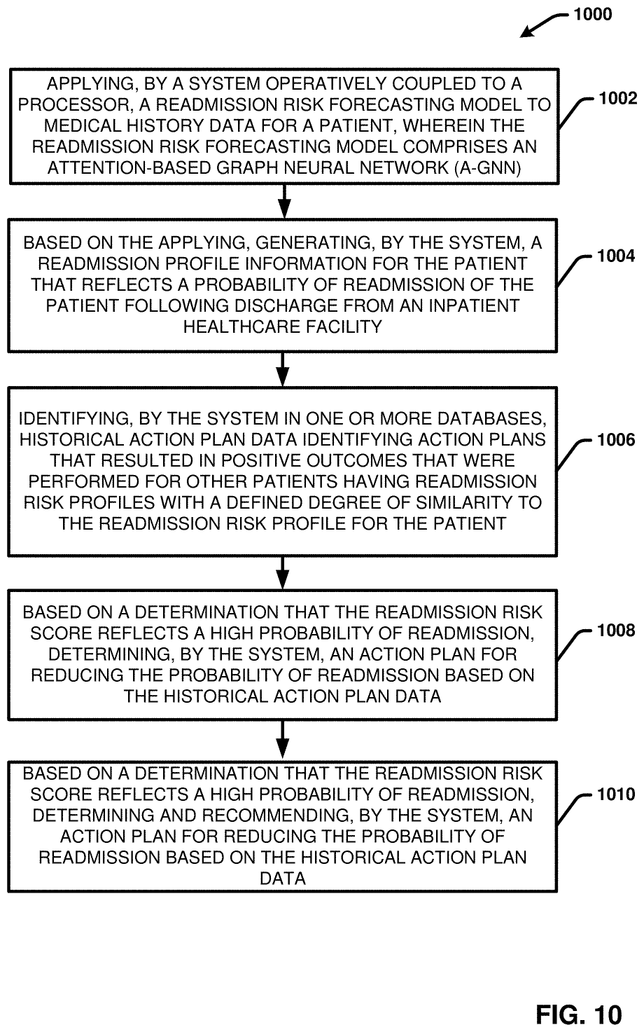

FIG. 10 presents a high-level flow diagram of another example computer implemented method 1000 for reducing unplanned readmissions in accordance with one or more embodiments of the disclosed subject matter. Repetitive description of like elements employed in respective embodiments is omitted for sake of brevity.

At 1002, a system operatively coupled to a processor (e.g., system 100, system 800, or the like), can apply (e.g., using readmission risk forecasting component 806) a readmission risk forecasting model (e.g., readmission risk forecasting model 106) to medical history data for a patient (e.g., patient data 102), wherein the readmission risk forecasting model comprises an A-GNN. At 1004, based on the applying, the system can generate (e.g., (e.g., by the readmission risk forecasting component 806) a readmission profile information for the patient (e.g., readmission profile information 114) for the patient that reflects a probability of readmission of the patient following discharge from an inpatient healthcare facility. At 1006, the system can identify (e.g., using similar case identification component 812) in one or more databases (e.g., at least one database comprising historical care plan data 120), historical action plan data identifying action plans that resulted in positive outcomes that were performed for other patients having readmission risk profiles with a defined degree of similarity to the readmission risk profile for the patient. At 1008, based on a determination that the readmission risk score reflects a high probability of readmission (e.g., relative to a defined threshold probability), the system can determine and recommend an action plan for reducing the probability of readmission based on the historical action plan data (e.g., via action plan generation component 814 and recommendation component 816).

It should be noted that, for simplicity of explanation, in some circumstances the computer-implemented methodologies are depicted and described herein as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts can be required to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

FIG. 11 can provide a non-limiting context for the various aspects of the disclosed subject matter, intended to provide a general description of a suitable environment in which the various aspects of the disclosed subject matter can be implemented. FIG. 11 illustrates a block diagram of an example, non-limiting operating environment in which one or more embodiments described herein can be facilitated. Repetitive description of like elements employed in other embodiments described herein is omitted for sake of brevity.

With reference to FIG. 11, a suitable operating environment 1100 for implementing various aspects of this disclosure can also include a computer 1102. The computer 1102 can also include a processing unit 1104, a system memory 1106, and a system bus 1108. The system bus 1108 couples system components including, but not limited to, the system memory 1106 to the processing unit 1104. The processing unit 1104 can be any of various available processors. Dual microprocessors and other multiprocessor architectures also can be employed as the processing unit 1104. The system bus 1108 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MCA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 11124), and Small Computer Systems Interface (SCSI).

The system memory 1106 can also include volatile memory 1110 and nonvolatile memory 1112. The basic input/output system (BIOS), containing the basic routines to transfer information between elements within the computer 1102, such as during start-up, is stored in nonvolatile memory 1112. Computer 1102 can also include removable/non-removable, volatile/non-volatile computer storage media. FIG. 11 illustrates, for example, a disk storage 1114. Disk storage 1114 can also include, but is not limited to, devices like a magnetic disk drive, floppy disk drive, tape drive, Jaz drive, Zip drive, LS-100 drive, flash memory card, or memory stick. The disk storage 1114 also can include storage media separately or in combination with other storage media. To facilitate connection of the disk storage 1114 to the system bus 1108, a removable or non-removable interface is typically used, such as interface 1116. FIG. 11 also depicts software that acts as an intermediary between users and the basic computer resources described in the suitable operating environment 1100. Such software can also include, for example, an operating system 1118. Operating system 1118, which can be stored on disk storage 1114, acts to control and allocate resources of the computer 1102.

System applications 1120 take advantage of the management of resources by operating system 1118 through program modules 1122 and program data 1124, e.g., stored either in system memory 1106 or on disk storage 1114. It is to be appreciated that this disclosure can be implemented with various operating systems or combinations of operating systems. A user enters commands or information into the computer 1102 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1104 through the system bus 1108 via interface port(s) 1130. Interface port(s) 1130 include, for example, a serial port, a parallel port, a game port, and a universal serial bus (USB). Output device(s) 1134 use some of the same type of ports as input device(s) 1136. Thus, for example, a USB port can be used to provide input to computer 1102, and to output information from computer 1102 to an output device 1134. Output adapter 1128 is provided to illustrate that there are some output devices 1134 like monitors, speakers, and printers, among other output devices 1134, which require special adapters. The output adapters 1128 include, by way of illustration and not limitation, video and sound cards that provide a means of connection between the output device 1134 and the system bus 1108. It should be noted that other devices and/or systems of devices provide both input and output capabilities such as remote computer(s) 1140.

Computer 1102 can operate in a networked environment using logical connections to one or more remote computers, such as remote computer(s) 114. The remote computer(s) 1140 can be a computer, a server, a router, a network PC, a workstation, a microprocessor based appliance, a peer device or other common network node and the like, and typically can also include many or all of the elements described relative to computer 1102. For purposes of brevity, only a memory storage device 1142 is illustrated with remote computer(s) 1140. Remote computer(s) 1140 is logically connected to computer 1102 through a network interface 1138 and then physically connected via communication connection 1132. Network interface 1138 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1132 refers to the hardware/software employed to connect the network interface 1138 to the system bus 1108. While communication connection 1132 is shown for illustrative clarity inside computer 1102, it can also be external to computer 1102. The hardware/software for connection to the network interface 1138 can also include, for exemplary purposes only, internal and external technologies such as, modems including regular telephone grade modems, cable modems and DSL modems, ISDN adapters, and Ethernet cards.

One or more embodiments described herein can be a system, a method, an apparatus and/or a computer program product at any possible technical detail level of integration. The computer program product can include a computer readable storage medium (or media) having computer readable program instructions thereon for causing a processor to carry out aspects of one or more embodiment. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. In this regard, in various embodiments, a computer readable storage medium as used herein can include non-transitory and tangible computer readable storage mediums.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of one or more embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of one or more embodiments.

Aspects of one or more embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and other devices to function in a particular manner, such that the computer readable storage medium having instructions stored therein comprises an article of manufacture including instructions which implement aspects of the function/act specified in the flowchart and block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and block diagram block or blocks.

The flowchart and block diagrams in the Figures illustrate the architecture, functionality, and operation of possible implementations of systems, methods, and computer program products according to various embodiments described herein. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and flowchart illustration, and combinations of blocks in the block diagrams and flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on one or more computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices. For example, in one or more embodiments, computer executable components can be executed from memory that can include or be comprised of one or more distributed memory units. As used herein, the term “memory” and “memory unit” are interchangeable. Further, one or more embodiments described herein can execute code of the computer executable components in a distributed manner, e.g., multiple processors combining or working cooperatively to execute code from one or more distributed memory units. As used herein, the term “memory” can encompass a single memory or memory unit at one location or multiple memories or memory units at one or more locations.

As used in this application, the terms “component,” “system,” “platform,” “interface,” and the like, can refer to and can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and a computer. By way of illustration, both an application running on a server and the server can be a component. One or more components can reside within a process or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that can provide specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result (e.g., including employing ML and/or AI techniques to determine the intermediate results), etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to: sensors, antennae, audio and/or visual output devices, other devices, etc.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches, and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
applying, by a system operatively coupled to a processor, applying a risk model on application specific retrospective data from at least one source, wherein the risk model comprises an attention-based graph neural network (A-GNN);
based on the applying, generating, by the system, an application specific risk score; and
facilitating providing, by the system, the application specific risk score to one or more entities.

2. The method of claim 1, wherein the risk model comprises a readmission risk forecasting model and the retrospective data comprises medical history data for a patient, wherein the application specific risk score comprises a readmission risk score for the patient that reflects a probability of readmission of the patient following discharge from an inpatient healthcare facility, and wherein the one or more entities comprise the patient or a clinician involved in the patients care.

3. The method of claim 2, further comprising, based on the applying:
identifying, by the system, one or more factors included in the medical history data for the patient that contribute to the readmission score; and
generating, by the system, importance scores for the one or more factors representing their degree of contribution to the readmission risk score.

4. The method of claim 3, wherein the one or more factors comprise a plurality of factors and wherein the method further comprises, based on the applying:
identifying, by the system, relationships between the factors that contribute to the readmission score.

5. The method of claim 4, further comprising, based on the applying:
generating, by the system, an interactive feature graph comprising nodes respectively corresponding to the factors and connections between the nodes representing the relationships.

6. The method of claim 5, further comprising:
facilitating rending, by the system, the interactive feature in a network accessible graphical user interface.

7. The method of claim 2, further comprising:
applying, by the system, an outlier detection model to the medical history data to determine whether the medical history data is within a scope of training data used to train the readmission risk forecasting model; and
generating, by the system, a warning notification based on a determination that the medical history data is outside the scope of the training data.

8. The method of claim 7, wherein the outlier detection model comprises another attention-based graph neural network (A-GNN).

9. The method of claim 2, wherein the readmission risk forecasting model comprises a machine learning model trained on historical medical history data for patients previously readmitted to one or more inpatient healthcare facilities following discharge.

10. The method of claim 3, further comprising:
recommending, by the system, an action plan for reducing the probability of readmission based on a determination that the readmission risk score reflects a high probability of readmission.

11. The method of claim 10, further comprising:
generating, by the system, a readmission risk profile for the patient comprising the readmission risk score, the one or more factors, and the importance scores;
identifying, by the system in one or more databases, historical action plan data identifying action plans that resulted in positive outcomes that were performed for other patients having readmission risk profiles with a defined degree of similarity to the readmission risk profile for the patient; and
determining, by the system, the action plan based on the historical action plan data.

12. The method of claim 11, wherein the identifying the historical action plan data is further based on the other patients having similar medical health histories to the patient.

13. The method of claim 11, wherein the determining the action plan further comprises employing one or more machine learning models.

14. A system, comprising:
a memory that stores computer executable components; and
a processor that executes the computer executable components stored in the memory, wherein the computer executable components comprise:
a readmission risk forecasting component that applies a risk forecasting model to medical history data for a patient and generates a readmission risk score for the patient that reflects a probability of readmission of the patient following discharge from an inpatient healthcare facility, wherein the readmission risk forecasting model comprises an attention-based graph neural network (A-GNN); and
a rendering component that facilitates providing the readmission risk score to at least one of the patient or a clinician involved in care of the patient.

15. The system of claim 14, wherein based on application of the risk forecasting model to medical history data for a patient, the readmission risk forecasting component further identifies one or more factors included in the medical history data for the patient that contribute to the readmission score, and generates importance scores for the one or more factors representing their degree of contribution to the readmission risk score.

16. The system of claim 14, wherein the one or more factors comprise a plurality of factors and wherein based on application of the risk forecasting model to medical history data for a patient, the readmission risk forecasting component further identifies relationships between the factors that contribute to the readmission score.

17. The system of claim 16, wherein the computer executable components further comprise:
a mapping component that generates an interactive feature graph comprising nodes respectively corresponding to the factors and connections between the nodes representing the relationships, and wherein the rendering component further facilitates rendering the interactive feature in a network accessible graphical user interface.

18. The system of claim 15, wherein the computer executable components further comprise:
a model scoping component that applies an outlier detection model to the medical history data to determine whether the medical history data is within a scope of training data used to train the readmission risk forecasting model; and
a notification component that generates a warning notification based on a determination that the medical history data is outside the scope of the training data.

19. The system of claim 15, wherein the readmission risk forecasting model comprises a machine learning model trained on historical medical history data for patients previously readmitted to one or more inpatient healthcare facilities following discharge.

20. The system of claim 16, wherein the computer executable components further comprise:
a recommendation component that recommends an action plan for reducing the probability of readmission based on a determination that the readmission risk score reflects a high probability of readmission.

21. The system of claim 20, wherein the risk forecasting component further generates a readmission risk profile for the patient comprising the readmission risk score, the one or more factors, and the importance scores, and wherein the computer executable components further comprise:
a similar case identification component that identifies, in one or more databases, historical action plan data identifying action plans that resulted in positive outcomes that were performed for other patients having readmission risk profiles with a defined degree of similarity to the readmission risk profile for the patient; and
an action plan generation component that determines the action plan based on the historical action plan data.

22. The system of claim 21, wherein the similar case identification component further identifies the historical action plan data based on the other patients having similar medical health histories to the patient.

23. The system of claim 21, wherein the action plan generation component further determines the action plan using one or more machine learning models.

24. A machine-readable storage medium, comprising executable instructions that, when executed by a processor, facilitate performance of operations, comprising:
applying a readmission risk forecasting model to medical history data for a patient, wherein the readmission risk forecasting model comprises an attention-based graph neural network (A-GNN);
based on the applying, generating a readmission risk score for the patient that reflects a probability of readmission of the patient following discharge from an inpatient healthcare facility; and
facilitating providing the readmission risk score to at least one of the patient or a clinician involved in care of the patient.
